(19) United States
(12) Patent Application Publication
(10) Pub. No.: US 2025/0000076 A1
(43) Pub. Date: Jan. 2, 2025

(54) SELF-ADJUSTING APPLICATOR

(71) Applicant: PowerPollen. Inc., Ames, IA (US)

(72) Inventors: Jason Cope, Ankeny, IA (US); Jason Kelsick, Elkhart, IA (US); Christopher Wendt, Monroe, IA (US)

(73) Assignee: PowerPollen. Inc., Ames, IA (US)

(21) Appl. No.: 18/606,699

(22) Filed: Mar. 15, 2024

Related U.S. Application Data
(60) Provisional application No. 63/524,385, filed on Jun. 30, 2023.

(51) Int. Cl. A01M 7/00; A01C 23/00

(52) U.S. Cl. CPC A01M 7/0057; A01C 23/008

(57) ABSTRACT

Provided is a self-adjusting applicator apparatus and method for use in applying solid, particulate compositions or liquid compositions to plants. In particular, the applicator is self-adjusting on a row of plants regardless of any curvature or inconsistency in plant row spacing. The applicator may include a biasing member, at least one pivot member, and at least one guide member. Contact between the at least one guide member and a plant may be configured to cause the at least one pivot member to pivot about a pivot point. The application means may be directly or indirectly attached to the at least one pivot member, such that the application means moves to best position itself about the plant to which material will be applied, thereby ensuring proper alignment of the application with a row of plants.

### CROSS REFERENCE TO RELATED APPLICATIONS

This application claims priority from U.S. Provisional Application Ser. No. 63/524,385 filed Jun. 30, 2023, and entitled SELF-ADJUSTING APPLICATOR FOR ROW CROPS. The contents of U.S. Provisional Application Ser. No. 63/524,385 are hereby incorporated in their entirety by reference.

### FIELD OF THE INVENTION

This invention relates generally to self-adjusting applicator apparatuses and their use in dispensing and applying materials in agricultural settings, such as traditional fields used for row crops. In particular, this invention discloses an applicator apparatus that self-centers on one or more rows of plants. The apparatus can be used for dispensing both dry and liquid materials.

### BACKGROUND

The current invention has application to the field of pollination and other crop production practices, including but not limited to seed, grain, ornamental, and fruit production practices. During the growth period of many crops, various materials are applied to the crop. Such materials can include liquids, such as fertilizers, pesticides, fungicides, or other liquid treatments. In addition, the materials can include dry materials in a particulate or powdered format, such as fertilizers, pesticides, or pollen. In all cases, materials being applied to crops may need to be applied to particular parts of the crop or at a particular height on the plant. The materials may need to be applied to flowers, leaves, fruits, roots, stems, stalks or other specific plant parts and structures.

The current invention is particularly suited to the intentional application of pollen, for example to plants that are receptive to pollen. Intentional pollination is used to produce specific hybrid seeds or plants, to pollinate plants when natural pollination has failed or is particularly poor, to supplement natural pollination, and/or to pollinate plants as needed for any other reason. In such circumstances, pollen must be applied to plants in such a manner to ensure that the pollen reaches the stigma of the receptive flowers. The targeted application of pollen requires an applicator apparatus that ensures the pollen is not harmed during the delivery, and that the pollen is applied at the correct location and in the correct density or amount.

As used in this disclosure, “pollen” may refer to either pure pollen or it can refer to pollen that has been mixed with other additives. Furthermore, the pollen may be freshly-collected pollen, recently-collected pollen, or pollen that was previously collected and subjected to short-term or long-term storage, including pollen that has been maintained at cool or cold temperatures, including cryopreserved pollen. When additives are combined with the pollen, they may be mixed with the pollen at specified additive to pollen ratios. The additives may be non-living particles, living particles, or they may be a combination of living and non-living particles. The additives may include, for example, diluents, anti-flocculants, absorbent materials, materials intended to separate particles, or be materials with other uses such as, but not limited to, additives that help maintain the viability of the pollen or other living particles.

It is important to prevent physical stress to pollen and other living particulates during handling, particularly when recalcitrant pollen is being used, or when using other living particulates, the viability of which is impacted by stressors such as heat, humidity, friction, crowding, compression, or other stressors. In particular, handling of the pollen by means of vibration, forced air, rotation, physical force or other means can be disruptive to pollen membrane integrity, at least in part as a result of the friction experienced during the distribution process. Accordingly, some embodiments of the present invention seek to minimize the stress applied to pollen, other living particulates, or other non-living particulate matter being distributed in a growing environment or applied to a crop. In some cases, additives may be used to reduce the impact of stressors. For example, additives may be used to reduce the effects of high humidity, thereby reducing clumping or aggregation of living or non-living particles. Research has demonstrated that live pollen grains that come into contact with the contents of burst dead pollen cells are adversely affected by such contact, reducing viability and killing the pollen cells (U.S. patent application Ser. No. 16/028,626). As such, pollen particles may be blended with additives to reduce contact with dead pollen contents. Other living particles that can be applied using the invention may include actual insects, such as those used in biological control regimes to combat crop pests or plant diseases, which may be gently blended with additives to improve their viability, prevent damage to the insects, to absorb waste products, and to provide nutrition during storage. Other living particles that can be applied using the invention may include bacteria, fungi, or virus particles that are being applied to treat crop pests or plant diseases. Bacteria and fungi, for example, may be blended with an additive that includes supporting nutrients for growth, that reduces damage, or otherwise protects the particles or improves their viability. In all cases, both living and non-living particles of all kinds may also be blended with additives that serve as diluents to improve the efficacy of delivering a metered dose to the crop.

Although straight rows are typically the goal when planting row crops, they are not often a reality. This is often due to severe weather events which compromises the plant or variations in the terrain on which the planting occurs but can be due to many other reasons, such as operator error when driving though a field, any of which can cause variations in any direction, including horizontal, vertical, and diagonal directions. These variations may also cause inconsistent plant spacing. However, as described above, precise application of materials is necessary and/or desirable. Because the precise application of materials must occur on rows of plants that may not be perfectly straight or on perfectly level ground, there is a need for self-adjusting application devices that precisely apply material on the individual plants within a row regardless of curves or other changes in row direction, plant height, and regardless of the plant spacing from one row to the next, or from one plant to the next within a row. As used in this specification, a material is a substance or mixture of substances that constitutes an object. Materials within the scope of the specification can be pure or impure, living or non-living matter.

### BRIEF DESCRIPTION OF THE FIGURES

FIG. 1 is a perspective view of a first embodiment of an apparatus of the present invention.

FIG. 2 is another perspective view of the apparatus of FIG. 1.

FIG. 3 is a perspective view of the apparatus of FIG. 2 in a field of a first type of plants.

FIG. 4 is a perspective view of the apparatus of FIG. 2 in a field of a second type of plants.

FIG. 5 is a rear perspective view of an apparatus head of the present invention.

FIG. 6 is a front perspective view of an apparatus head of the present invention.

FIG. 7 is a front elevation view of an apparatus head of the present invention.

FIG. 8 is a front elevation view of the self-adjusting pivot of the present invention.

FIG. 9 is a front perspective view of an apparatus head of the present invention, showing the head tilting in a first direction.

FIG. 10 is a front perspective view of an apparatus head of the present invention showing the head tilting in a second direction.

FIG. 11 is a perspective view of the connection between at least one pivot member and the pivot point of an apparatus of the present invention.

### SUMMARY OF THE INVENTION

Provided are embodiments of a self-adjusting plant row guide for applying material via at least one applicator to a plurality of plants in a row. The row may include a longitudinal center, and at least one of the plants may be located a lateral distance from the longitudinal center. The self-adjusting plant row guide may comprise at least one guide member configured to successively sense each of the plurality of plants in the row. If a plant is located a different lateral distance from the longitudinal center than the immediate previously sensed plant, the guide member actuates the self-adjusting plant row guide to move the at least one applicator.

In some embodiments, the self-adjusting plant row guide includes a self-adjusting pivot, which may include a pivot point, a biasing member, and at least one pivot member. The biasing member may bias the self-adjusting plant row guide in a neutral or balanced position. The neutral or balanced position may be at the longitudinal center of the row. The at least one guide member may sense the plants by touching the plants. The touching may cause the at least one guide member to move in a lateral position, such as following an arcuate path outward and upward from the neutral or balanced position. The movement of the guide member may overcome the bias of the biasing member. This may cause the at least one pivot member to pivot about the pivot point. In some embodiments, the plant row guide may include two pivot members.

The self-adjusting plant row guide may include at least one applicator, and the pivot member may be engaged with the applicator such that movement of the pivot member may move the at least one applicator. The applicator may be a nozzle, atomizer, tube or one of many other forms of application systems which are known to those skilled in the art. Further, the applicator may be designed for metered application. The self-adjusting plant row guide may be configured to apply pollen. It may include at least one height adjustment member configured to adjust the height of the at least one guide member with respect to the ground.

In a first embodiment of the invention, a self-adjusting applicator is provided comprising a biasing member, at least one pivot member, and at least one guide member. Contact between the guide member and a plant is configured to overcome the biasing member, causing the at least one pivot member to pivot about a pivot point. Some embodiments of the invention may include means for applying material to the plant. Such a means may include, but is not limited to, a nozzle and an atomizer. The applicator may be designed for metered application and/or configured to apply liquids, solids, and or powders. For example, the applicator may be designed to apply pollen and/or pesticide. In addition, the invention may include at least one height adjustment member.

The height adjustment member can be used to effectively position the applicator. The type of applicator to be used may be selected based on its intended use, the plant species, and the material to be applied. The height adjustment will allow for the applicator to be positioned at the desired level with respect to the ground or with respect to the height of the plants to which the material is being applied. In some embodiments, the material is being applied at ground level, and in such cases may be targeting the plant roots. In other embodiments, the material is being applied to other plant tissues, including, but not limited to, the stem, the leaves, the flower buds, the closed or open flowers, specific reproductive structures (such as stigmas, corn silks, etc.), the fruits, the developing seeds, or any other plant morphological tissue or structure. The self-centering row guide provides the opportunity for highly customized and targeted application of material to plants or the soil through the combination of the right type of applicator type, the specific material being applied, and the application occurring at the right height, along with the self-centering capability of the row guide being used to correctly position the plants at the time of application.

Embodiments of the invention may be adapted for use in one or more of a field, greenhouse, vertical farming facility, hoop house, and a high tunnel. Further embodiments of the invention may be adapted to be propelled by means selected from the group consisting of manpower, all-terrain vehicle, tractor, a robotic applicator, motor vehicle, or a stationary applicator.

### DETAILED DESCRIPTION

Example embodiments will now be described more fully with reference to the accompanying drawings. Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers and/or sections should not be limited by these terms. These terms may be only used to distinguish one element, component, region, layer or section from another element, component, region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Disclosed is a unique self-adjusting apparatus for use in agricultural applications, including, but not limited to, for the application of material to row crops. Unlike existing devices for application in the field, the disclosed applicator has the ability to shift on a per-plant basis to accommodate centering on the plants within a row. The self-adjustment feature keeps the apparatus centered on a row of plants such that any applications being made are delivered effectively and in the proper location. This feature enables true centering on rows that are inconsistently spaced and which curve or otherwise deviate from perfectly straight. In addition, the self-adjusting feature allows for true centering on rows even when the plant centering point within the row varies.

In the United States, the typical desired plant spacing from one row to the next in conventional agricultural field cropping systems is 30 inches. In other countries, or in other types of agricultural cropping systems, plant row spacing can vary significantly, but in general, most row crop systems attempt to provide an even row spacing. In reality, the spacing from one row to the next can vary for a number of different reasons. In some cases, the topography of the land where the planting is done can impact the row spacing because during the same planting pass, some plants may be planted on a relatively flat surface while others are on a slope. The rows on the slope will naturally have a different spacing from the rows planted on the flat surface. In other cases, when a tractor makes a turn and begins a new row, the outer planted row on the new set of rows may not be at a uniform spacing from the outer planted row on the previously planted set of rows. In some cases, there may be obstacles in a field that must be avoided, thereby causing variation in row spacing. In other cases, severe weather, including wind and hail, may compromise the plant's ability to stand up completely straight.

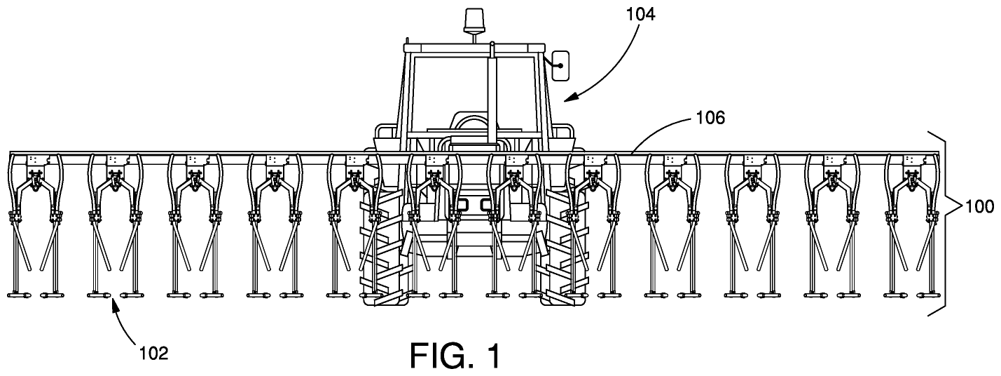

In a first embodiment of the invention, a self-adjusting applicator 100 is provided, as shown in FIG. 1. In the illustrated example, the self-adjusting applicator 100 is adapted for attachment to field machinery 104, such as a tractor. However, the self-adjusting applicator 100 may be adapted for any method of moving plants through the apparatus, including but not limited to manpower, all-terrain vehicle (ATV), robotic applicator, other field machinery, aerial machinery, or a stationary applicator wherein plants or portions thereof are moved through the applicator by a conveyor or other mechanism. In one embodiment, the self-adjusting applicator 100 may be attached to a vehicle driven by an operator. In another embodiment, the self-adjusting applicator 100 may be attached to an autonomous vehicle, such as a self-driving car, a self-driving tractor, or a self-piloting drone. The self-adjusting applicator 100 may include at least one head 102. In some embodiments adapted for attachment to field machinery 104, the self-adjusting applicator includes a plurality of heads 102. The illustrated example in FIG. 1 includes twelve heads 102. Each head 102 is configured to receive at least one plant. In some embodiments, each head 102 receives a plurality of plants in succession as the applicator 100 moves through one or more rows of plants. In some embodiments, the heads 102 are attached to the field machinery 104 via one or more supports, such as a support network 106, which one of skill in the art may modify based on factors including, but not limited to, number of heads 102, type of field machinery 104 or other propelling force, and type of plant.

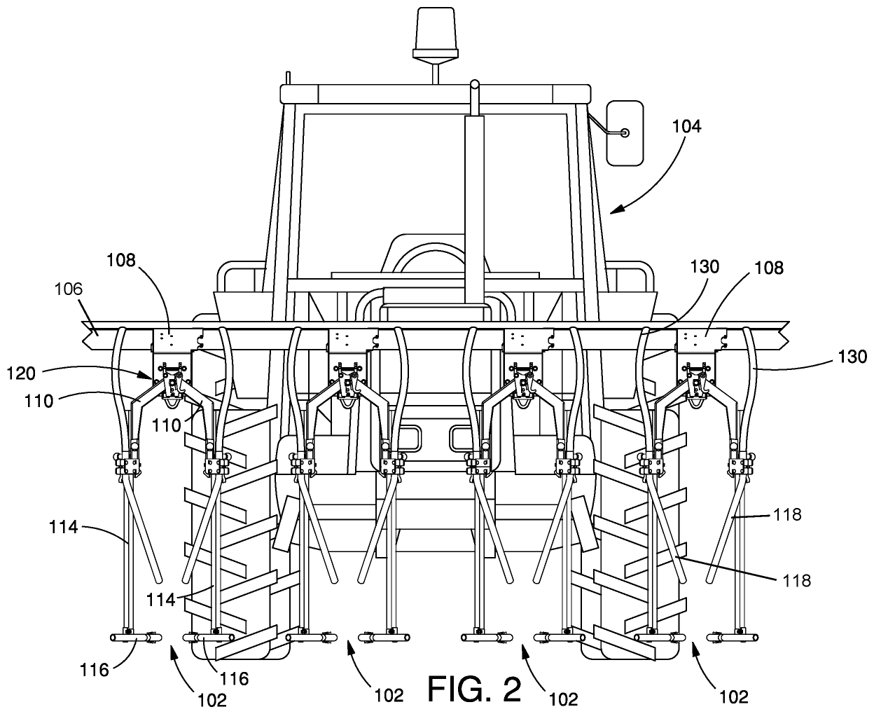

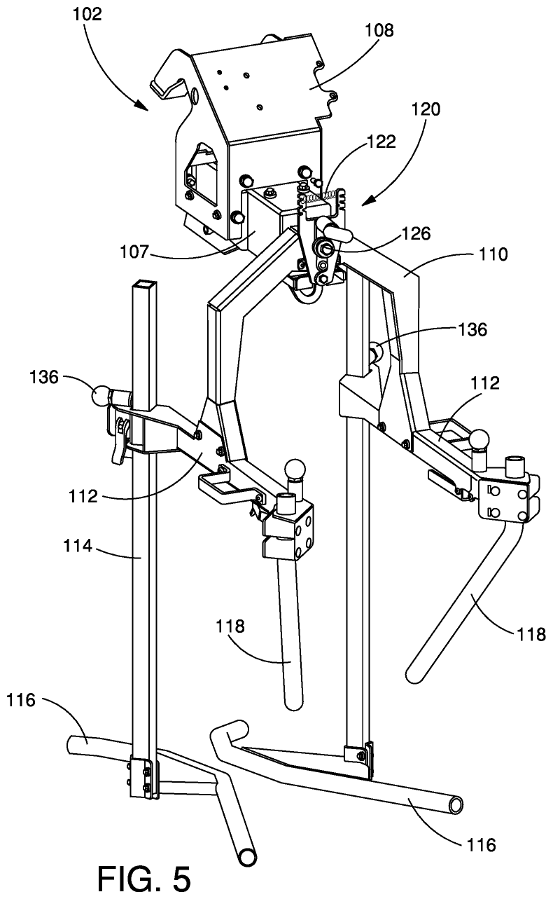

Turning to FIG. 2, a further view of a first embodiment of a self-adjusting applicator 100 is provided. Again, the self-adjusting applicator 100 is adapted for attachment to field machinery 104. The illustrated embodiment further shows four heads 102 of the present invention mounted to the support network 106 of the present invention. Each head 102 is configured to receive at least one plant, such as a plurality of plants in succession as the self-adjusting applicator moves through one or more rows of plants. In the illustrated embodiment of the present invention, each head 102 of the present invention comprises a bracket 108. The bracket 108 connects the head 102 of the present invention to the support network 106, such as via a bracket projection 107 (best shown in FIG. 5). The head 102 further comprises one or more pivot members 110, which in the illustrated embodiment are pivoting arms attached to the bracket projection 107 at the first end, which is illustrated as the top of the pivot members 110. Although the pivot members are shown as arms in the illustrated embodiment, it will be understood by one of skill in the art that any shape, size, and/or configuration of pivot member(s) may be used without departing from the scope of the invention. Also shown are adjustable length members 114 and applicator tips 118. One or more guide members 116 are attached to the adjustable length members 114, such as at the bottom of the adjustable length members 114. The invention includes a self-adjusting pivot 120. The self-adjusting pivot 120 enables the pivot members 110, adjustable length legs 114, guide bars 116, and applicator tips 118 (and other components discussed hereinbelow) to pivot laterally with respect to a neutral or balanced position in order to maintain alignment with a plant and/or plant row. In some embodiments, such a neutral or balanced position may correspond to the longitudinal center of a row of crops.

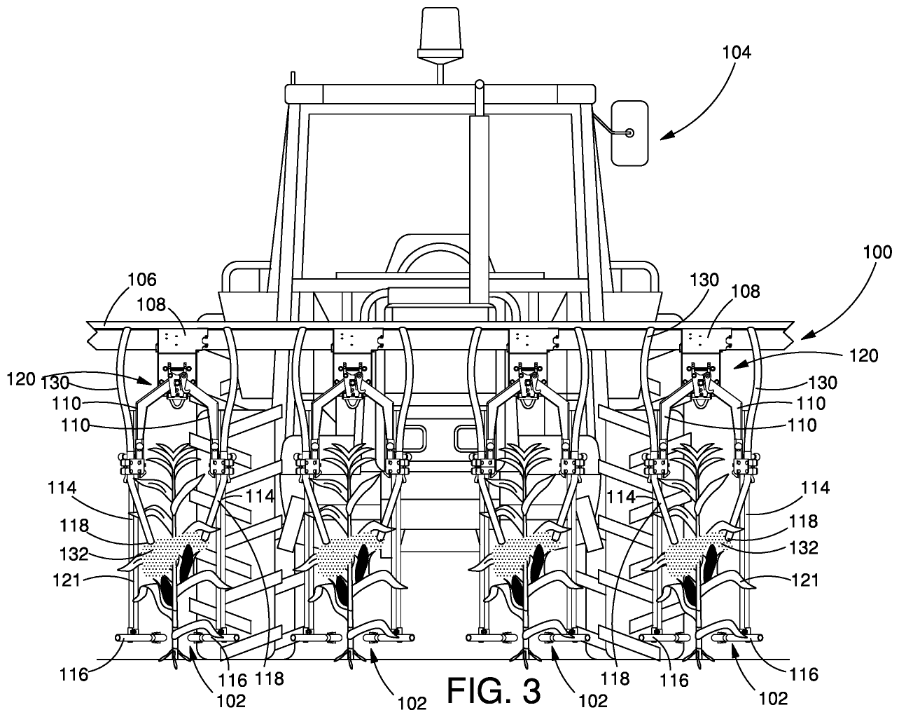

Turning to FIG. 3, an embodiment of the present invention is shown in use on a row of corn plants 121, which is a relatively tall row crop. Other tall row crops include, but are not limited to, sorghum, cotton, canola, cannabis, sunflowers, and trees, including but not limited to, young trees. The self-adjusting applicator 100 is shown attached to a piece of field machinery 104. The self-adjusting applicator 100 comprises at least one head 102. FIG. 3 shows four heads 102. In the illustrated embodiment of the present invention, each head 102 of the present invention includes a bracket 108. Each head 102 further includes a self-adjusting pivot 120 which enables at least a portion of the head to pivot in a lateral direction. Each head 102 may further include pivot members 110 connected to the self-adjusting pivot 120. Each head 102 may further include applicator tips 118. In the illustrated embodiment, the applicator tips 118 are shown dispensing a cloud of pollen 132.

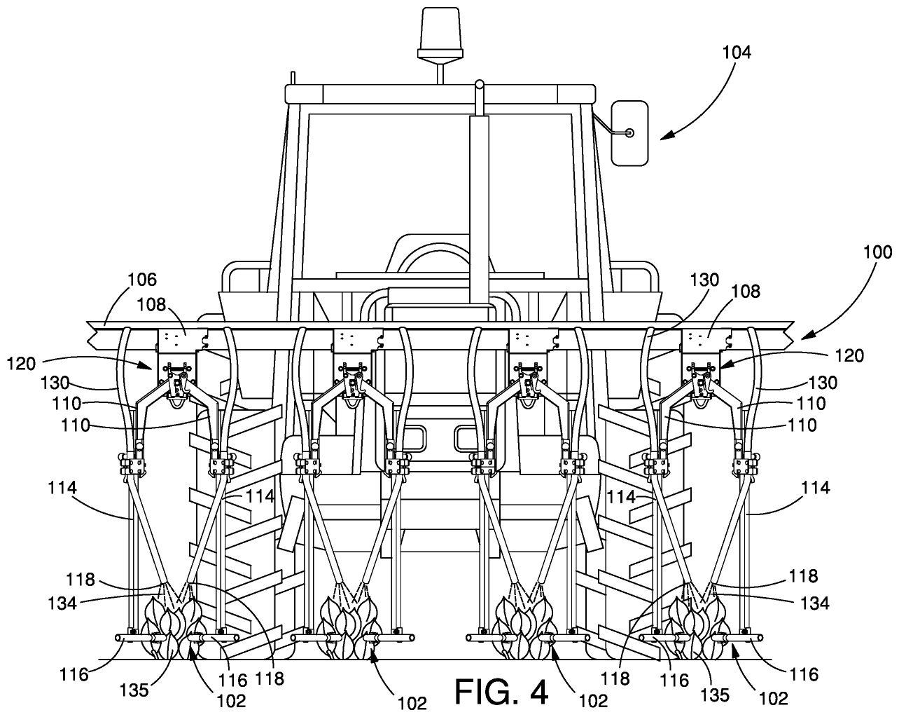

Turning to FIG. 4, an additional embodiment of the present invention is shown in use on a row of short plants 135. Examples of relatively shorter plants that may be used in connection with the invention include, but are not limited to, young plants of any kind, soybeans, alfalfa, cereal crops such as wheat, barley, pearl millet, and oats, ornamental plants, and rice. The self-adjusting applicator 100 is shown attached to a piece of field machinery 104. The self-adjusting applicator 100 comprises at least one head 102. In the illustrated embodiment four heads 102 are shown. Each head 102 further includes pivot members 110 connected to a self-adjusting pivot 120. The self-adjusting pivot 120 enables at least a portion of the head to pivot, such as in an arc. Each head 102 may further include applicator tips 118 attached to the pivot members 110. In the illustrated embodiment, the applicator tips 118 are shown dispensing a liquid spray 134 on to the plants 135. The liquid spray 134 could be, but is not limited to, a pesticide, such as a fungicide, an insecticide, or an herbicide.

Turning to FIG. 5, a view of a head 102 of a self-adjusting applicator 100 is shown. The head includes a bracket 108 which connects the head 102 to a support network 106 (shown in FIGS. 1-4). The bracket 108 includes a bracket projection 107. Attached to the bracket projection 107 is one or more pivot members 110. The illustrated embodiment includes two pivoting arms. In the illustrated embodiment, each of said pivot members 110 is attached to one or more braces 112 at the end opposite the bracket projection 107. The illustrated embodiment includes one brace 112 per pivot member 110. Moreover, the illustrated brace 112 can be described as a generally horizontal bar. However, one of skill in the art will recognize that any size, shape, or type of brace may be used. In addition, the brace is optional, as the components held or supported by the brace could be held or supported directly by the one or more pivot members. The braces 112 each hold one or more adjustable length members 114. Attached to the adjustable length members 114 is at least one guide member 116. In the illustrated embodiment, a pair of guide members 116 extend both forward and rearward (referring to the direction of forward travel) to guide the pivoting members 110 towards a plant or plant row. The guide members 116 are configured to accept one or more plants. In the illustrated embodiments, a set of guide members 116 accepts a series of plants in succession that are planted in a row. Accordingly, the guide bars 116 guide the pivoting members 110 towards the plants as the plants pass between the guide bars 116. In the illustrated embodiment, one or more applicator tips 118 are shown attached to the brace 112 at the opposite end from the adjustable length members 114, although any configuration may be used without departing from the scope of the invention.

The illustrated embodiment also includes a self-adjusting pivot 120. As will be described in detail below, the self-adjusting pivot 120 enables the pivot members 110 to pivot with respect to an arch pivot point 126. In cases where the row spacing is variable, the self-adjusting pivot 120 described herein allows the head 102, including components such as the pivot members 110 and the applicator tips 118 to move into a position that accommodates the variability. Additionally, the self-adjusting pivot 120 accommodates rows with curves or other variations within the planting row. This ensures that the applicator tips 118 remain properly positioned relative to the plant regardless of any variability in the row.

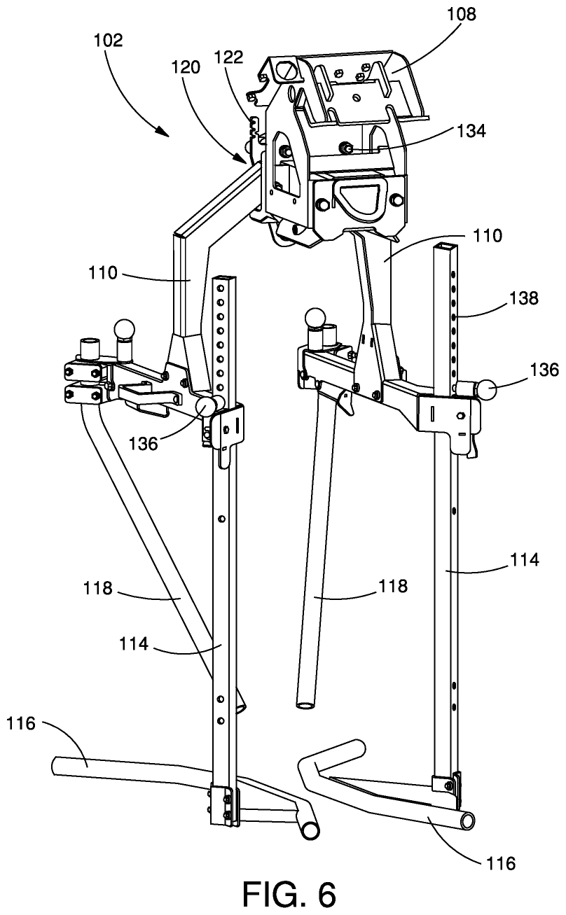

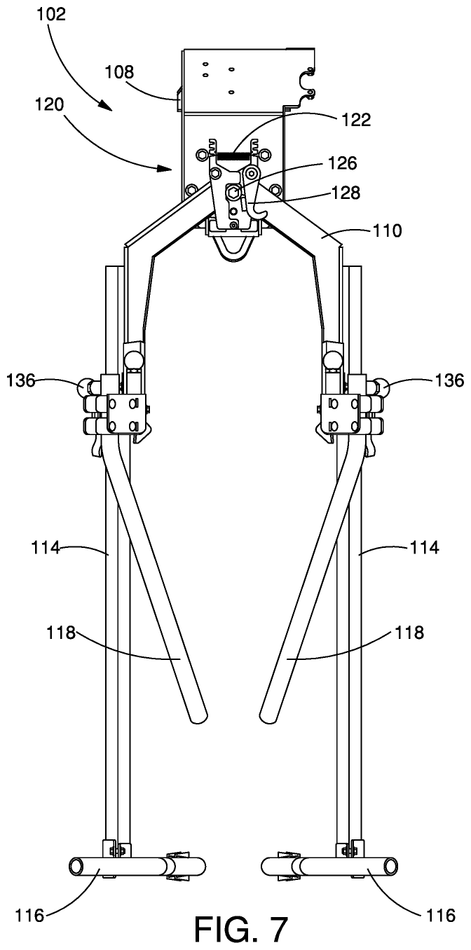

Shown in FIG. 6 is a rear view of a head 102 of the present invention. A rear view of the bracket 108 is shown. In some embodiments, the bracket 108 is configured to attach the head 102 to the support network 106. The head 102 includes a self-adjusting pivot 120 attached to the bracket 108 (shown best in FIGS. 7-10). In the shown embodiment, the self-adjusting pivot may be secured using a connection such as bolts 134. As will be understood by one of skill in the art, any type of connection may be used. The head 102 further comprises pivot members 110 connected to the bracket 108 by the self-adjusting pivot 120, as described in further detail below. The head may further comprise adjustable length members 114. In the illustrated embodiment, the length of the adjustable length members 114 is adjusted using a pin adjuster 136 in combination with a plurality of spaced holes 138 along the length of the adjustable length members 114. However, in other embodiments, members that are not capable of length adjustment may be included.

The adjustable length members 110 may have a first and a second end, said first end being closer to said bracket 108 and said second end being further from said bracket 108. Said first end may also be referred to as the proximal end and said second end may also be referred to as the distal end. Although the leg adjustment mechanism has been described as a pin adjuster 136 being used with a plurality of spaced holes 138, one having at least ordinary skill in the art will be able to substitute this adjustment method with any number of alternative mechanisms without departing from the scope of the invention. The head 102 may further comprise applicator tips 118 attached to the pivot members 110. The head 102 may further comprise guide members 116 fixed to the distal end of the adjustable length members 114. In the illustrated embodiment, the guide members 116 are shaped in a manner so that the opening between the guide bars 116 is broader at the front so as to find plants that are far from center.

Shown in FIG. 7 is a head 102 of a self-adjusting applicator 100 of the present invention. The head 102 in the illustrated embodiment includes a bracket 108 attached to a self-adjusting pivot 120 via a bracket projection (shown in FIG. 5). The self-adjusting pivot 120 may further include at least one locking member 128. In some embodiments, the locking member 128 may be in the form of an immobilizing latch. In other embodiments, the locking member 128 could be a pneumatic mechanism, pinned mechanism, or sliding barrier mechanism. The self-adjusting pivot 120 is actuated by an adjusting mechanism. In the illustrated embodiment, the adjusting mechanism is a biasing member, such as a spring 122. In other embodiments, the biasing member may be a gas cylinder, elastic plastic member, rubber member, or a dampener. The head further comprises one or more pivot members 110 connected to the self-adjusting pivot 120. In the illustrated embodiment, adjustable length members 114 and applicator tips 118 are also included. The adjustable length members 114 may be adjusted by a pin adjuster 136. The adjustable length members 114 may connect to one or more guide members 116 attached to the lower portion of the adjustable length members 114.

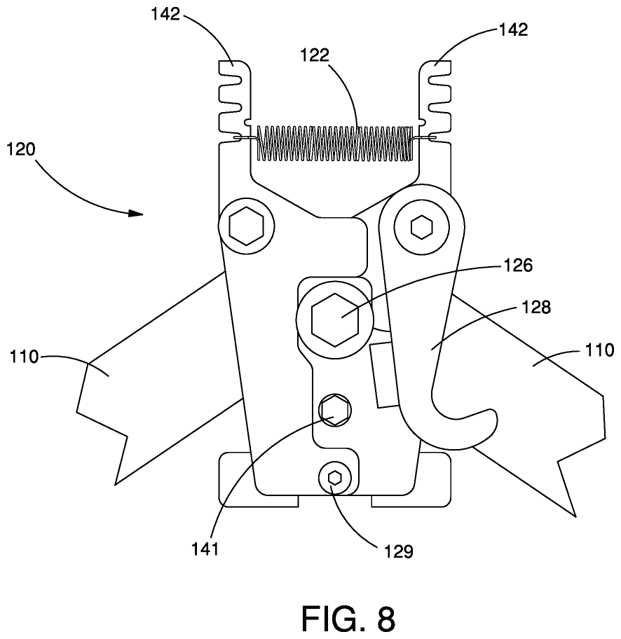

Shown in FIG. 8 is an embodiment of a self-adjusting pivot 120 of the present invention. This illustrated design includes a biasing member, for example a spring 122. The illustrated embodiment does not require electrical, hydraulic, or pneumatic components, although such components could be included without departing from the scope of the invention. This results in an efficient design that is less vulnerable to component failure. The self-adjusting pivot shown further provides an arch pivot point 126 about which the pivot members 110 can pivot. The illustrated embodiment further provides a locking member 128 to secure the pivot members 110 to prevent undesirable movement of the pivot members 110, such as during transport of the self-adjusting applicator 100. Also shown in FIG. 8 are two biasing member prongs 142 which are connected at the top by the spring 122, and at the bottom by a biasing member pivot point 129 which goes though the bottom of both biasing member prongs 142. Above the biasing member pivot point 129 is an actuating pin 141 which is connected to the pivot members 110 and configured to move with them.

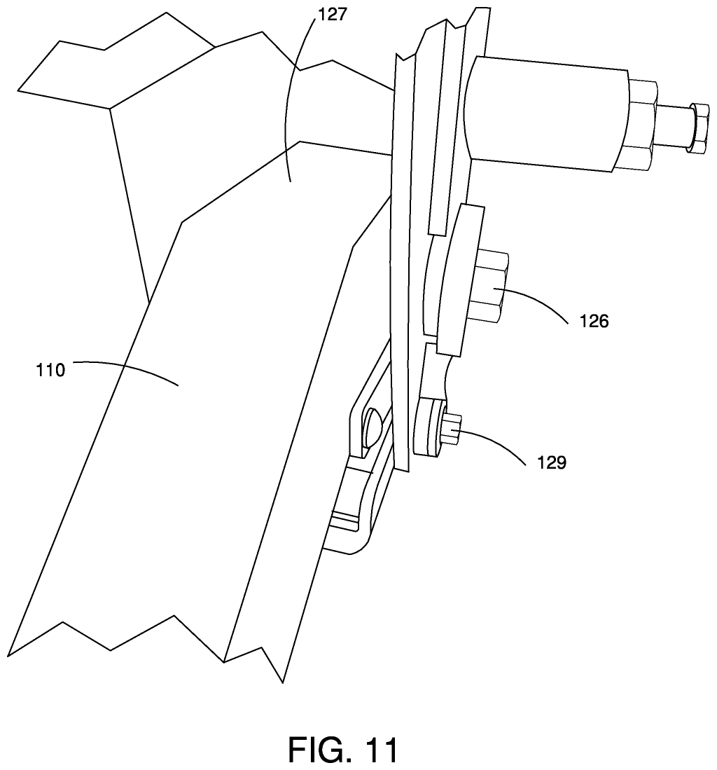

Turning to FIG. 11, a figure showing the interior of a self-adjusting pivot 120 of the present invention is shown. FIG. 11 shows the top of a pivot member 110. Also shown is a bridge 127 that connects the two pivot members 110. The second pivot member is not shown in FIG. 11, but in the illustrated embodiment, the two pivot members 110 and bridge 127 are symmetrical and form one continuous component. The arch pivot point 126 extends through the bridge 127. In some embodiments, the resistance between the two pivot members 110 is balanced at the arch pivot point 126. Force in either direction, which will be discussed in detail below, will cause the pivot members 110 to respond and move accordingly. In the illustrated embodiment, the pivot members 110 and bridge 127 create a pendulum that moves in an arcuate path.

Operation of the illustrated embodiment of the invention will now be described. In operation, the self-adjusting applicator 100 travels through a plurality of rows of crops, such as a field. For example, FIGS. 1-4 show a plurality of heads secured to a piece of field machinery 104, such as a tractor. As the field machinery moves through the plurality of rows of crops, the plurality of heads 102 are preferably configured such that the applicators each correspond to one row of plants. Typically the rows are immediately next to each other, but other configurations may be used. As noted in detail above, the individual rows may vary in their path and may not be straight. Focusing on an individual head 102, the guide members 116 will contact successive plants in a row. In some embodiments, the guide members 116 together create a funnel shape. However, rather than funneling the plant to the applicator head, the applicator head moves to accommodate the plant, thereby being as gentle as possible with the plants. In some embodiments, the funnel shape may be used to funnel plants that are bushier together in a more concentrated space for application of material. In other embodiments, the funnel shape does not bring portions of the plant together in a smaller space. Although the biasing member 122 biases the head 102 in a particular, balanced position, such as centered, the biasing member 122 could be configured so that the balanced position is shifted either to the right or the left from a front view. The guide member 116 senses a plant. In the illustrated embodiment, the sensing occurs via touch or contact of the guide member(s) 116 with the plant. This contact overcomes the bias of the biasing member 122 and causes the head 102 to pivot about the arch pivot point 126 in the direction necessary to self-center the head 102 with respect to the plant. This results in accurate application of material via the applicator tips 118 with respect to the plant. As will be appreciated by one of skill the art, the applicator may include a system for delivery of material to the applicator tips 118. In the illustrated embodiment, a material delivery network runs along and/or within the support network 106, bracket 108, bracket projection 107, pivot members 110, and/or brace 112. Moreover, the height of the guide members 116 may be adjusted to adapt to a plurality of heights as needed, such as via the illustrated pin adjuster 136.

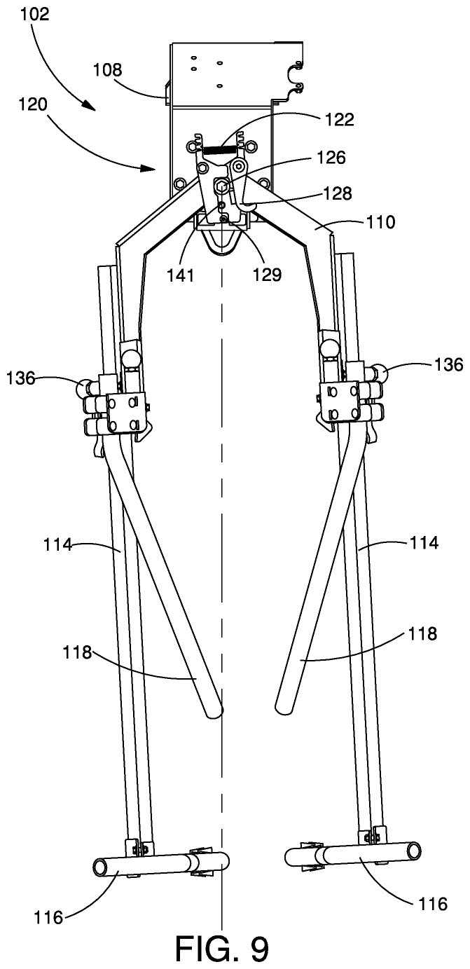

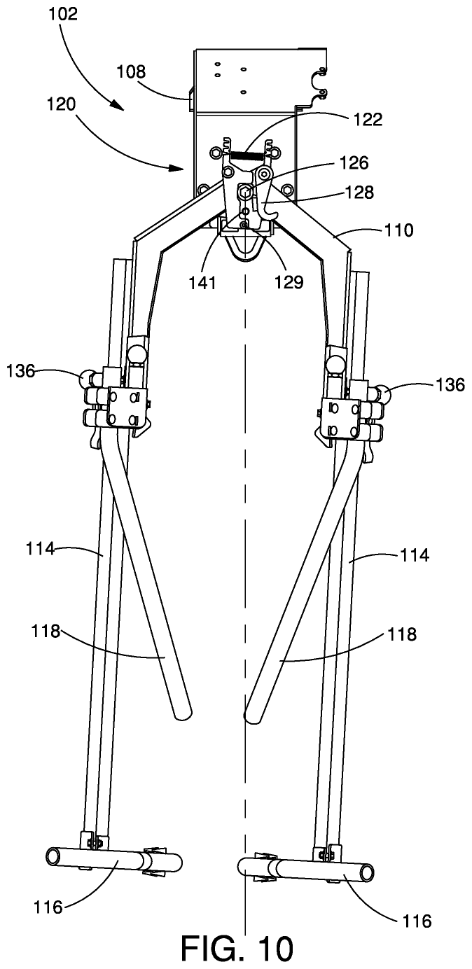

FIGS. 9-10 illustrate operation of a head 102 of a self-adjusting applicator 100 of the present invention. The head 102 may include a bracket 108 attached to a self-adjusting pivot 120 via a bracket projection 107. The self-adjusting pivot 120 may further include at least one locking member 128. The locking member 128 may be in the form of an immobilizing latch. The self-adjusting pivot 120 includes a biasing member, such as a spring 122. The spring 122 is attached to two biasing member prongs 142 to enable the operation of the biasing member. In FIG. 9, the self-adjusting pivot 120 is tilted towards the right relative to the balanced position when viewed from the front, movement which would have been actuated when the guide members 116 came into contact with a plant that was to the right of center 140. FIG. 10, on the other hand, illustrates the self-adjusting pivot 120 tiled towards the left when viewed from the front, movement which would have been actuated when the guide members 116 came into contact with a plant that was to the left of center 140. More specifically, in the illustrated embodiment, the biasing member prongs 142 are both connected to the biasing prong pivot point 129, as well as to the spring 122. Accordingly, when the pivot members 110 are shifted to the right as seen in FIG. 9, the actuating pin 141 comes into contact with the right biasing member prong 142, causing the right biasing member prong 142 to move away from the left biasing member prong 142, tensioning the spring 122. When the force acting on the guide members 110 is no longer present, the spring 122 will contract and force the right biasing member prong 142 back towards the left prong 142, returning the pivot members 110 to their neutral position. In FIG. 10, the applied force moves the actuating pin 141 into contact with the left biasing member prong 142. This creates a similar effect, where the spring 122 will return both itself and the pivot members 110 to a neutral position after the applied force is no longer present. The head further comprises one or more pivot members 110 connected to the self-adjusting pivot 120. The adjustable length members 114 may be adjusted by a pin adjuster 136.

This invention can be used to apply products to any species of crop plant that can be accommodated by the device to which the applicator is affixed. In general, the applicator is designed for use on plants that are grown in rows. It can be adapted to operate in any environment including, but not limited to, ideal or target outdoor growing environments, off-season environments, or controlled environments (e.g. shade/glass/green/hoop houses, growth chambers, vertical farming facilities, hydroponic facilities, aeroponic facilities etc.).

The apparatus of the invention can be affixed to a variety of vehicles allowing it to travel across rows of plants. The apparatus can be mounted to manual delivery vehicles or robots that might be used in indoor environments or smaller plot sizes. The apparatus can also be mounted to field driven machinery.

The applicator can be adapted to apply solid (particulate) materials, such as powders or pollen, but can also be adapted to apply liquid materials. One of skill in the art will recognize that the tubing to supply the product to be applied can vary to suit the properties of the applied product. In addition, different types of nozzles known in the art and different nozzle apertures can be used at the end of the applicator tubes to apply the product. Non-limiting examples of nozzles include single-fluid nozzles or two-fluid nozzles, including configurations where the two-fluid nozzle distributes a liquid by means of an atomizing gas, such as compressed air or other gases, as well as electric, pneumatic, or hydraulically actuated or controlled nozzles.

The applicator can be used to provide intentional pollination to improve overall pollination events in crops that are typically self-pollinated, which can be useful when the crop's level of pollen production is unexpectedly reduced or when other conditions threaten the success of the typical self-pollination outcome. It can also be used to provide intentional pollination in crops that are typically not self-pollinated, thereby providing a means of conducting cross-pollinations. In either case, the applicator can be used to dispense freshly collected pollen or previously collected and preserved pollen.

While the applicator of the present invention is particularly suited to row crops, it also has utility for dispensing materials onto plants that have tillers that share a common root structure, such as rice and wheat, or plants that have more of a branching bush shape, such as soybeans. When used to dispense materials on those types of plants, the guide members serve to gather or bunch the plants together to concentrate their biomass for better application of materials. This decreases necessary application volumes as well as increasing success rate in pollinations.

Although various representative embodiments of this invention have been described above with a certain degree of particularity, those skilled in the art could make numerous alterations to the disclosed embodiments without departing from the spirit or scope of the inventive subject matter set forth in the specification and claims. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements, and/or substantial equivalents.

1. A self-adjusting plant row guide apparatus for applying material via at least one applicator to a plurality of plants in a row, said row having a longitudinal center and wherein at least one of said plants is located a lateral distance from said longitudinal center, said self-adjusting plant row guide apparatus comprising at least one guide member configured to successively sense each of said plurality of plants in said row and if a plant is located a different lateral distance from said longitudinal center than the immediate previously sensed plant, said guide member actuates said self-adjusting plant row guide apparatus to move said at least one applicator.

2. The self-adjusting plant row guide apparatus of claim 1 further comprising a self-adjusting pivot.

3. The self-adjusting plant row guide apparatus of claim 2 wherein said self-adjusting pivot comprises:
a. a pivot point,
b. a biasing member,
c. and at least one pivot member.

4. The self-adjusting plant row guide apparatus of claim 3 wherein said biasing member biases said self-adjusting plant row guide in a neutral position.

5. The self-adjusting plant row guide apparatus of claim 4 wherein said neutral position is at the longitudinal center of said row.

6. The self-adjusting plant row guide apparatus of claim 4 wherein said at least one guide member senses said plants by touching said plants.

7. The self-adjusting plant row guide apparatus of claim 6 wherein the touching of said at least one guide member by said plants causes said at least one guide member to move laterally.

8. The self-adjusting plant row guide apparatus of claim 7 wherein the lateral movement of said at least one guide member overcomes the bias of said biasing member, causing movement of said biasing member.

9. The self-adjusting plant row guide apparatus of claim 8 wherein the movement of said biasing member causes said at least one pivot member to pivot about said pivot point.

10. The self-adjusting plant row guide apparatus of claim 9 wherein said at least one pivot member is engaged with said at least one applicator, such that movement of said at least one pivot member moves said at least one applicator.

11. The self-adjusting plant row guide apparatus of claim 10 comprising two pivot members.

12. The self-adjusting plant row guide apparatus of claim 11 wherein each pivot member is connected to at least one applicator.

13. The self-adjusting plant row guide apparatus of claim 12, wherein said applicator is selected from the group consisting of:
a. a nozzle
b. an atomizer

14. The self-adjusting plant row guide apparatus of claim 12, wherein said applicator is designed for metered application.

15. The self-adjusting plant row guide apparatus of claim 12, wherein said applicator is configured to apply pollen.

16. The self-adjusting plant row guide apparatus of claim 10 further comprising at least one height adjustment member configured to adjust the height of said at least one guide member with respect to the ground.

17. A method of applying material to a plurality of plants using a self-adjusting plant row guide apparatus having at least one guide member, wherein said self-adjusting plant row guide apparatus is connected to at least one applicator, comprising;
a. Sensing said plurality of plants with said at least one guide member connected to said at least one applicator, wherein said sensing causes said self-adjusting plant row guide to move to accommodate said plurality of plants, and
b. Applying material to said plurality of plants with said at least one applicator after sensing said plurality of plants with said guide member.

18. The method of claim 17 wherein said plurality of plants are selected from the group consisting of corn, sorghum, cotton, canola, sunflowers, soybeans, wheat, barley, pearl millet, oats, and rice.

19. The method of claim 17 wherein said sensing further comprises bunching said plurality of plants by said guide member.

20. The method of claim 17 wherein said material is selected from the group consisting of solid particulates and liquids.

21. The method of claim 20 wherein said solid particulates are selected from the group of powdered fertilizers, powdered pesticides or combinations thereof.

22. The method of claim 20 wherein said liquids are selected from the group of liquid fertilizers, liquid pesticides, liquid fungicides, or combinations thereof.

23. The method of claim 17 wherein said material is pollen.

24. The method of claim 17 further comprising adjusting the height of said at least one guide member to target application of said material to a particular plant tissue of said plurality of plants.

25. The method of claim 24 wherein said particular plant tissue is selected from the group consisting of one or more stems, one or more leaves, one or more flower buds, one or more closed flowers, one or more open flowers, one or more stigmas, one or more fruits, or one or more developing seeds.

26. The method of claim 24 wherein said particular plant tissue comprises one or more corn silks.
